(19) United States
(12) Patent Application Publication
(10) Pub. No.: US 2005/0106101 A1
(43) Pub. Date: May 19, 2005

(54) Novel chemical agents comprising an adenosine moiety or an adenosine analog moiety and an imaging moiety and methods of their use

(76) Inventors: Ajay Purohit, Sudbury, MA (US); David S. Casebier, Carlisle, MA (US)

Correspondence Address: STEPHEN B. DAVIS;BRISTOL-MYERS SQUIBB COMPANY, PATENT DEPARTMENT, P O BOX 4000, PRINCETON, NJ, 08543-4000, US

(21) Appl. No.: 10/974,223

(22) Filed: Oct. 27, 2004

Related U.S. Application Data
(60) Provisional application No. 60/516,564, filed on Oct. 31, 2003.

(51) Int. Cl. A61K 51/00; C07H 19/22; C07D 473/12

(52) U.S. Cl. 424/1.73; 534/11; 536/27.1; 544/277

(57) ABSTRACT

The present invention is directed to novel chemical agents for compounds and their use for imaging myocardial perfusion. The invention also is directed to a kit for forming such novel agents. The chemical agents for the present invention comprising (a) an adenosine analog moiety or an adenosine moiety, and (b) an imaging moiety.

### CROSS REFERENCE TO RELATED APPLICATION

This non-provisional application claims priority from provisional application U.S. Ser. No. 60/516,564, filed Oct. 31, 2003.

### FIELD OF THE INVENTION

The present invention relates to novel chemical agents comprising (1) an adenosine moiety or an adenosine analog moiety and (2) an imaging moiety; and their use for diagnosing certain disorders in a subject. The present invention further relates to a kit for myocardial perfusion imaging comprising (1) a compound A comprising an adenosine moiety or an adenosine analog moiety and (2) a compound B comprising an imaging moiety; wherein compounds A and B can be reacted with each other to form an imaging agent.

### BACKGROUND OF THE INVENTION

Adenosine is known to accumulate in myocardial tissues, and is used to induce coronary artery vasodilatation in conjunction with myocardial perfusion imaging. In such cases, adenosine is typically administered first, followed by administration of an imaging compound, such as a radioactive ion.

Certain ¹⁸F based adenosine derivatives have been used for imaging cancer, for example, 2′-Fluoro 2′deoxy adenosine (Kim, C. G et. Al., J. Pharm Sci. 1996, 85, 339-344), however, to the best of Applicants' knowledge, no adenosine-based agents have been developed for myocardial imaging.

### SUMMARY OF THE INVENTION

The present invention relates to novel chemical agents comprising (1) an adenosine moiety or an adenosine analog moiety and (2) an imaging moiety. In one embodiment, the novel chemical agent is a chemical compound comprising an adenosine moiety or an adenosine analog moiety covalently linked to an imaging moiety, either directly or indirectly via a linker moiety. In another embodiment, the novel chemical agent is a chemical complex comprising an adenosine moiety or an adenosine analog moiety linked to an imaging moiety via a non-covalent force. Non-limiting examples of the non-covalent force include ionic, hydrogen bonding, and van der Waals force. Such agent are useful as imaging agents.

The present invention is also directed to a method of imaging myocardial perfusion. Such method comprises administering to a subject an imaging agent which comprises (1) an adenosine moiety or an adenosine analog moiety and (2) an imaging moiety; and scanning the subject using diagnostic imaging to detect areas of greater imaging moiety concentration. In one embodiment, the imaging agent is a chemical compound comprising an adenosine moiety or an adenosine analog moiety covalently linked to an imaging moiety, either directly or indirectly via a linker moiety. In another embodiment, the novel chemical agent is a chemical complex comprising an adenosine moiety or an adenosine analog moiety linked to an imaging moiety via a non-covalent force. Non-limiting examples of the non-covalent force include ionic, hydrogen bonding, and van der Waals force.

The present invention is further directed to a kit for myocardial perfusion imaging. Such kit comprises (1) a compound A which comprises an adenosine moiety or an adenosine analog moiety and (2) a compound B which comprises an imaging moiety. Compounds A and B can be reacted to each other and form an imaging agent which comprises (1) an adenosine moiety or an adenosine analog moiety and (2) an imaging moiety. In one embodiment, the imaging agent is a chemical compound comprising an adenosine moiety or an adenosine analog moiety covalently linked to an imaging moiety, either directly or indirectly via a linker moiety. In another embodiment, the novel chemical agent is a chemical complex comprising an adenosine moiety or an adenosine analog moiety linked to an imaging moiety via a non-covalent force. Non-limiting examples of the non-covalent force include ionic, hydrogen bonding, and van der Waals force.

### DETAILED DESCRIPTION OF THE INVENTION

The present invention utilizes adenosine's affinity for myocardium and combines one or more adenosine analog moieties with one or more imaging moieties to form a novel agent suitable for imaging myocardial perfusion.

The present invention relates to novel chemical agents comprising an adenosine analog moiety and an imaging moiety. In one embodiment, the novel chemical agent is a chemical compound comprising an adenosine analog moiety covalently linked to an imaging moiety, either directly or indirectly via a linker moiety. In another embodiment, the novel chemical agent is a chemical complex comprising an adenosine analog moiety linked to an imaging moiety via a non-covalent force. Non-limiting examples of the non-covalent force include ionic, hydrogen bonding, and van der Waals force.

The present invention is also directed to a method of imaging myocardial perfusion. Such method comprises administering to a subject an imaging agent which comprises an adenosine analog moiety and an imaging moiety; and scanning the subject using diagnostic imaging to detect areas of greater adenosine analog moiety concentration. In one embodiment, the imaging agent is a chemical compound comprising an adenosine analog moiety covalently linked to an imaging moiety, either directly or indirectly via a linker moiety. In another embodiment, the novel chemical agent is a chemical complex comprising an adenosine analog moiety linked to an imaging moiety via a non-covalent force. Non-limiting examples of the non-covalent force include ionic, hydrogen bonding, and van der Waals force.

The present invention is further directed to a kit for myocardial perfusion imaging. Such kit comprises (1) a compound A which comprises an adenosine moiety or an adenosine analog moiety and (2) a compound B which comprises an imaging moiety. Compounds A and B can be reacted to each other and form an imaging agent which comprises (1) an adenosine moiety or an adenosine analog moiety and (2) an imaging moiety. In one embodiment, the imaging agent is a chemical compound comprising an adenosine moiety or an adenosine analog moiety covalently linked to an imaging moiety, either directly or indirectly via a linker moiety. In another embodiment, the novel chemical agent is a chemical complex comprising an adenosine moiety or an adenosine analog moiety linked to an imaging moiety via a non-covalent force. Non-limiting examples of the non-covalent force include ionic, hydrogen bonding, and van der Waals force.

In a first embodiment, the imaging agent is described by Formula (I): 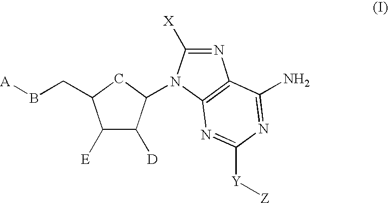
wherein
A is an imaging moiety or H;
B is a direct bond, O, alkylene, arylene, or alkylene ether;
C is O, N(R₁), or CH(R₁), wherein R₁ is H or C₁-C₆ alkyl;
D is an imaging moiety, F, OH, or H;
E is an imaging moiety, F, OH, or H;
X is an imaging moiety or H;
Y is a direct bond, alkylene, arylene, or alkylene ether; and
Z is an imaging moiety or H;
with the proviso that at least one of A, D, E, X, and Z is an imaging moiety.

In a first preferred embodiment of Formula (I), X is H.

In a second preferred embodiment of Formula (I), Y is a direct bond.

In a third preferred embodiment of Formula (I), B is a direct bond and A is an imaging moiety.

In a fourth preferred embodiment of Formula (I), B is O and A is H.

In a fifth preferred embodiment of Formula (I), B is an alkylene ether.

In a sixth preferred embodiment of Formula (I), the imaging agent is not [¹⁸F]fluorodeoxyadenosine.

In a more preferred embodiment of Formula (I), B is a C₁-C₆ alkylene ether.

In another more preferred embodiment of Formula (I), B is ethyl ether.

In a first most preferred embodiment of Formula (I),
A is ¹⁸F or OH;
B is ethyl ether;
C is O;
D and E are each OH;
X is H;
Y is a direct bond; and
Z is H or ¹⁸F,
   with the proviso that at least one of A and Z is ¹⁸F.

In this most preferred embodiment of Formula (I), the imaging moiety is ¹⁸F, which can be detected by nuclear medicine imaging, for example by positron emission tomography (“PET”). Advantageously, ¹⁸F will eventually decompose to ¹⁸O.

In a second most preferred embodiment of Formula (I), the imaging agent is:

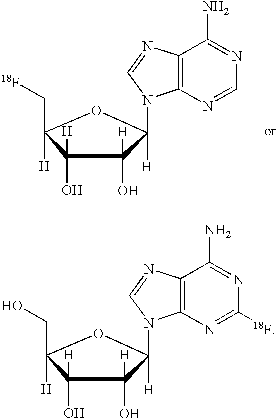

In a second embodiment, the imaging agent is described by Formula (II): 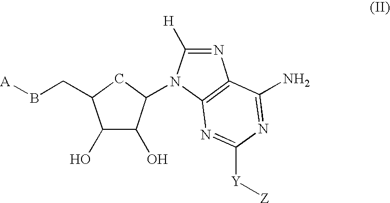
wherein
A is an imaging moiety or H;
B is a direct bond or O;
Y is a direct bond, alkylene, arylene, or alkylene ether; and
Z is an imaging moiety or H;
with the proviso that at least one of A and Z is an imaging moiety.

In a preferred embodiment of Formula (II), Y is a direct bond.

In a third embodiment, the imaging agent is described by Formula (III): 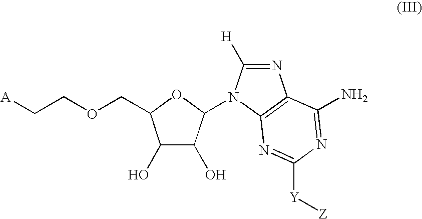
wherein
A is an imaging moiety or H;
Y is a direct bond, alkylene, arylene, or alkylene ether; and
Z is an imaging moiety or H;
with the proviso that at least one of A and Z is an imaging moiety.

In a preferred embodiment of Formula (III), Y is a direct bond.

Adenosine Analog Moieties

An adenosine analog moiety refers to a moiety that is considered, by one of ordinary skill in the art, as structurally similar to an adenosine moiety but differs slightly.

Imaging Moieties

Imaging moieties include those that are well known to those skilled in the art, and include those moieties that may be useful in the generation of diagnostic images by diagnostic techniques well known to the ordinarily skilled artisan. An imaging moiety is sometimes also referred to as a contrast moiety.

In one embodiment, the imaging moiety may be a radioisotope for nuclear medicine imaging, a radioisotope for X-ray CT imaging, a paramagnetic species for use in MRI imaging, an echogenic entity for use in ultrasound imaging, a fluorescent entity for use in fluorescence imaging, or an a light-active entity for use in optical imaging.

Nuclear medicine imaging moiety of the present invention include ¹¹C, ¹³N, ¹⁸F, ¹²³I, ¹²⁵I, ^{99m}Tc, ⁹⁵Tc, ¹¹¹In, ⁶²Cu, ⁶⁴Cu, ⁶⁷Ga, and ⁶⁸Ga. ¹¹C-Palmitate has been used to probe fatty acid oxidation and ¹¹C-acetate has been used to assess oxidative metabolism in the myocardium (Brown, M., Marshall, D. R., Sobel, B. E., Bergmann, S. R. Circulation, 1987, 76, 687-696). ¹³N-Ammonia has been used widely to image myocardial perfusion (Krivokapich J; Smith G T; Huang S C; Hoffman E J; Ratib O; Phelps M E; Schelbert H R. Circulation, 1989, 80, 1328-37). Compounds based on ¹⁸F have been used for imaging purpose for hypoxia and cancer (Pauwels, E. K. J., A. A. van der Klaau w., Corporaal, T., Stokkel, M. P. M. Drugs of the Future, 2002, 27, 655-667). 15-(p-(¹²³I)-iodophenyl)-pentadecanoic acid and 15-(p-(¹²³I)-iodophenyl)-3(R,S)-methylpentadecanoic acid are two iodinated agents that have been used for imaging myocardial metabolism. In one embodiment, the imaging moiety employed in the present imaging agents is ¹⁸F. Further imaging agents of the present invention may be comprised of one or more adenosine moieties or adenosine analog moieties attached to one or more X-ray absorbing or “heavy” atoms of atomic number 20 or greater, further comprising an optional linking moiety, L, between the one or more adenosine moieties or adenosine analog moieties and the X-ray absorbing atoms. A frequently used heavy atom in X-ray imaging agents is iodine. Recently, X-ray imaging agents comprised of metal chelates (Wallace, R., U.S. Pat. No. 5,417,959) and polychelates comprised of a plurality of metal ions (Love, D., U.S. Pat. No. 5,679,810) have been disclosed. More recently, multinuclear cluster complexes have been disclosed as X-ray imaging agents (U.S. Pat. No. 5,804,161, PCT WO91/14460, and PCT WO 92/17215). The disclosures of each of the foregoing documents are hereby incorporated herein by reference in their entireties. Preferred metals include Re, Sm, Ho, Lu, Pm, Y, Bi, Pd, Gd, La, Au, Au, Yb, Dy, Cu, Rh, Ag, and Ir.

MRI imaging agents of the present invention may be comprised of one or more adenosine moieties or adenosine analog moieties attached to one or more paramagnetic metal ions, further comprising an optional linking moiety, L, between the one or more adenosine moieties or adenosine analog moieties and the paramagnetic metal ions. The paramagnetic metal ions may be present in the form of metal chelates or complexes or metal oxide particles. U.S. Pat. Nos. 5,412,148, and 5,760,191, describe examples of chelators for paramagnetic metal ions for use in MRI imaging agents. U.S. Pat. No. 5,801,228, U.S. Pat. No. 5,567,411, and U.S. Pat. No. 5,281,704, describe examples of polychelants useful for complexing more than one paramagnetic metal ion for use in MRI imaging agents. U.S. Pat. No. 5,520,904, describes particulate compositions comprised of paramagnetic metal ions for use as MRI imaging agents. The disclosures of each of the foregoing documents are hereby incorporated herein by reference in their entireties. Preferred metals include Gd³⁺, Fe³⁺, In³⁺, and Mn²⁺.

The ultrasound imaging agents of the present invention may comprise one or more adenosine moieties or adenosine analog moieties attached to or incorporated into a microbubble of a biocompatible gas, a liquid carrier, and a surfactant microsphere, further comprising an optional linking moiety, L, between the one or more adenosine moieties or adenosine analog moieties and the microbubble. In this context, the term “liquid carrier” means aqueous solution and the term “surfactant” means any amphiphilic material which may produce a reduction in interfacial tension in a solution. A list of suitable surfactants for forming surfactant microspheres is disclosed, for example, in EP0727225A2, the disclosure of which is hereby incorporated herein by reference in its entirety. The term “surfactant microsphere” includes microspheres, nanospheres, liposomes, vesicles and the like. The biocompatible gas can be any physiologically accepted gas, including, for example, air, or a fluorocarbon, such as a C₃-C₅ perfluoroalkane, which provides the difference in echogenicity and thus the contrast in ultrasound imaging. The gas may be encapsulated, contained, or otherwise constrained in or by the microsphere to which is attached the analog moiety, optionally via a linking group. The attachment can be covalent, ionic or by van der Waals forces. Specific examples of suitable imaging moieties include, for example, lipid encapsulated perfluorocarbons with a plurality of tumor neovasculature receptor binding peptides, polypeptides or peptidomimetics. Examples of gas filled imaging moieties include those found in U.S. patent application Ser. No. 09/931,317, filed Aug. 16, 2001, and U.S. Pat. Nos. 5,088,499, 5,547,656, 5,228,446, 5,585,112, and 5,846,517, the disclosures of which are hereby incorporated herein by reference in their entireties.

Exemplary Methods of Making

### 5′-Fluoroadenosine

5′-fluoroadenosine can be made by selectively protecting the 2′ and 3′ hydroxyl's followed by activation of the 5′ alcohol and subsequently displacing it with a suitable source of F. This is then followed by the removal of the protecting groups.

### 2-Fluoroadenosine

2-fluoroadenosine can be made by first converting guanosine to 2-aminoadenosine, activating the 2 amino group followed by conversion to 2-fluoroadenosine.

### 5′-(2-fluoroethoxy)adenosine

5′-(2-fluoroethoxy)adenosine can be made by protecting the 2′ and 3′ and 5′ hydroxyl's, followed by protection of the N⁶ amine, deprotection of the 5′ hydroxyl and reacting with the corresponding activated fluoroalcohol and finally removing all protecting groups

The foregoing chemical transformations may be conducted using techniques which would be readily apparent to one of ordinary skill in the art, once armed with the teachings in the present applications. Preferred reaction solvents include, for example, DMF, NMP, DMSO, THF, EtOAc, DCM, and chloroform. The reaction solution may be kept neutral or basic by the addition of an amine such as triethylamine or DIEA. Reactions may be carried out at ambient temperatures and protected from oxygen and water with a nitrogen atmosphere.

Temporary protecting groups may be used to prevent other reactive functionality, such as amines, thiols, alcohols, phenols, and carboxylic acids, from participating in the reaction. Preferred amine protecting groups include, for example, t-butoxycarbonyl and trityl (removed under mild acidic conditions), Fmoc (removed by the use of secondary amines such as piperidine), and benzyloxycarbonyl (removed by strong acid or by catalytic hydrogenolysis). The trityl group may also used for the protection of thiols, phenols, and alcohols. Preferred carboxylic acid protecting groups include, for example, t-Butyl ester (removed by mild acid), benzyl ester (usually removed by catalytic hydrogenolysis), and alkyl esters such as methyl or ethyl (usually removed by mild base). All protecting groups may be removed at the conclusion of synthesis using the conditions described above for the individual protecting groups, and the final product may be purified by techniques which would be readily apparent to one of ordinary skill in the art, once armed with the present disclosure.

Use

The imaging agents of the present invention may be used in a method of imaging, including methods of imaging in a subject (e.g., a human patient or an animal) comprising administering the imaging agent to the subject by injection, infusion, or any other known method, and imaging the area of the subject wherein the event of interest is located.

The useful dosage to be administered and the particular mode of administration will vary depending upon such factors as age, weight, and particular region to be treated, as well as the particular imaging agent used, the diagnostic use contemplated, and the form of the formulation, for example, suspension, emulsion, microsphere, liposome, or the like, as will be readily apparent to those skilled in the art.

Typically, dosage is administered at lower levels and increased until the desirable diagnostic effect is achieved. In one embodiment, the above-described imaging agents may be administered by intravenous injection, usually in saline solution, at a dose of about 0.1 to about 100 mCi per 70 kg body weight (and all combinations and subcombinations of dosage ranges and specific dosages therein), or preferably at a dose of about 0.5 to about 50 mCi. Imaging is performed using techniques well known to the ordinarily skilled artisan.

For use as nuclear medicine imaging agents, the compositions of the present invention, dosages, administered by intravenous injection, will typically range from about 0.5 μmol/kg to about 1.5 mmol/kg (and all combinations and subcombinations of dosage ranges and specific dosages therein), preferably about 0.8 μmol/kg to about 1.2 mmol/kg.

For use as MRI imaging agents, the compositions of the present invention may be used in a similar manner as other MRI agents as described in U.S. Pat. No. 5,155,215; U.S. Pat. No. 5,087,440; Margerstadt et al., Magn. Reson. Med., 1986, 3, 808; Runge et al., Radiology, 1988, 166, 835; and Bousquet et al., Radiology, 1988, 166, 693. The disclosures of each of the foregoing documents are hereby incorporated herein by reference in their entireties. Generally, sterile aqueous solutions of the imaging agents may be administered to a patient intravenously in dosages ranging from about 0.01 to about 1.0 mmoles per kg body weight (and all combinations and subcombinations of dosage ranges and specific dosages therein).

The ultrasound imaging agents of the present invention may be administered by intravenous injection in an amount from about 10 to about 30 μL (and all combinations and subcombinations of dosage ranges and specific dosages therein) of the echogenic gas per kg body weight or by infusion at a rate of approximately 3 μL/kg/min.

Buffers can optionally be included. Buffers useful in the preparation of imaging agents and kits include, for example, phosphate, citrate, sulfosalicylate, and acetate buffers. A more complete list can be found in the United States Pharmacopoeia, the disclosure of which is hereby incorporated herein by reference in its entirety.

Lyophilization aids can optionally be included. Lyophilization aids useful in the preparation of imaging agents and kits include, for example, mannitol, lactose, sorbitol, dextran, FICOLL® polymer, and polyvinylpyrrolidine (PVP).

Stabilization aids can optionally be included. Stabilization aids useful in the preparation of imaging agents and kits include, for example, ascorbic acid, cysteine, monothioglycerol, sodium bisulfite, sodium metabisulfite, gentisic acid, and inositol.

Solubilization aids can optionally be included. Solubilization aids useful in the preparation of imaging agents and kits include, for example, ethanol, glycerin, polyethylene glycol, propylene glycol, polyoxyethylene sorbitan monooleate, sorbitan monoloeate, polysorbates, poly(oxyethylene)-poly(oxypropylene)-poly(oxyethylene) block copolymers (“Pluronics”) and lecithin. Preferred solubilizing aids are polyethylene glycol and Pluronics.

Bacteriostats can be optionally included. Bacteriostats useful in the preparation of imaging agents and kits include, for example, benzyl alcohol, benzalkonium chloride, chlorbutanol, and methyl, propyl, or butyl paraben.

A component in a diagnostic kit can also serve more than one function. For example, a reducing agent for a radionuclide can also serve as a stabilization aid, or a buffer can also serve as a transfer ligand, or a lyophilization aid can also serve as a transfer, ancillary, or co-ligand.

The compounds herein described may have asymmetric centers. Unless otherwise indicated, all chiral, diastereomeric and racemic forms are included in the present invention. Many geometric isomers of olefins, C═N double bonds, and the like can also be present in the compounds described herein, and all such stable isomers are contemplated in the present invention. It will be appreciated that compounds of the present invention contain asymmetrically substituted carbon atoms, and may be isolated in optically active or racemic forms. It is well known in the art how to prepare optically active forms, such as by resolution of racemic forms or by synthesis from optically active starting materials. Two distinct isomers (cis and trans) of the peptide bond are known to occur; both can also be present in the compounds described herein, and all such stable isomers are contemplated in the present invention. The D and L-isomers of a particular amino acid are designated herein using the conventional 3-letter abbreviation of the amino acid, as indicated by the following examples: D-Leu, or L-Leu.

When any variable occurs more than one time in any substituent or in any formula, its definition in each occurrence is independent of its definition at every other occurrence. Thus, for example, if a group, or plurality of groups, is shown to be substituted with 0-2 R, then said group(s) may optionally be substituted with up to two R, and R at each occurrence in each group is selected independently from the defined list of possible R. Also, by way of example, for the group —N(R′)₂, each of the two R′ substituents on N is independently selected from the defined list of possible R′. Combinations of substituents and/or variables are permissible only if such combinations result in stable compounds. When a bond to a substituent is shown to cross the bond connecting two atoms in a ring, then such substituent may be bonded to any atom on the ring.

Definitions

As used herein, “alkyl” is intended to include both branched and straight-chain saturated aliphatic hydrocarbon groups having the specified number of carbon atoms, examples of which include, but are not limited to, methyl, ethyl, n-propyl, i-propyl, n-butyl, i-butyl, sec-butyl, t-butyl, pentyl, hexyl, heptyl, octyl, nonyl, and decyl; cycloalkyl including saturated and partially unsaturated ring groups, including mono-, bi- or poly-cyclic ring systems, such as cyclopropyl, cyclobutyl, cyclopentyl, cyclohexyl, cycloheptyl, cyclooctyl and adamantyl; bicycloalkyl including saturated bicyclic ring groups such as [3.3.0]bicyclooctane, [4.3.0]bicyclononane, [4.4.0]bicyclodecane(decalin), [2.2.2]bicyclooctane, and so forth.

The term “alkoxy” means an alkyl-CO— group wherein alkyl is as previously described. Exemplary groups include methoxy, ethoxy, and so forth.

As used herein, the term “alkene” or “alkenyl” is intended to include hydrocarbon chains having the specified number of carbon atoms of either a straight or branched configuration and one or more unsaturated carbon-carbon bonds which may occur in any stable point along the chain, such as ethenyl, propenyl, and the like.

As used herein, the term “alkyne” or “alkynyl” is intended to include hydrocarbon chains having the specified number of carbon atoms of either a straight or branched configuration and one or more unsaturated carbon-carbon triple bonds which may occur in any stable point along the chain, such as propargyl, and the like.

As used herein, the term alkyl ether is intended to include hydrocarbon chains having the specified number of carbon atoms of either a straight or branched configuration and a noncarbon atom, such as S or O, with two points of attachment (i.e., is a diradical) in the chain.

As used herein, “aryl” or “aromatic residue” is intended to mean phenyl or naphthyl, which when substituted, the substitution can be at any position.

The term “aryloxy” means an aryl-CO— group wherein aryl is as previously described. Exemplary groups include phenoxy and naphthoxy.

As used herein, the term “alkaryl” means an aryl group bearing an alkyl group of 1-10 carbon atoms; the term “aralkyl” means an alkyl group of 1-10 carbon atoms bearing an aryl group; the term “arylalkaryl” means an aryl group bearing an alkyl group of 1-10 carbon atoms bearing an aryl group; the term “heteroaralkyl” means an alkyl group of 1-10 carbon atoms bearing an aryl group and a heteroatom; and the term “heterocycloalkyl” means an alkyl group of 1-10 carbon atoms bearing a heterocycle.

“Ancillary” or “co-ligands” are ligands that may be incorporated into a radiopharmaceutical during its synthesis. They may serve to complete the coordination sphere of the radionuclide together with the chelator or radionuclide bonding unit of the reagent. For radiopharmaceuticals comprised of a binary ligand system, the radionuclide coordination sphere may be composed of one or more chelators or bonding units from one or more reagents and one or more ancillary or co-ligands, provided that there are a total of two types of ligands, chelators or bonding units. For example, a radiopharmaceutical comprised of one chelator or bonding unit from one reagent and two of the same ancillary or co-ligands and a radiopharmaceutical comprised of two chelators or bonding units from one or two reagents and one ancillary or co-ligand are both considered to be comprised of binary ligand systems. For radiopharmaceuticals comprised of a ternary ligand system, the radionuclide coordination sphere may be composed of one or more chelators or bonding units from one or more reagents and one or more of two different types of ancillary or co-ligands, provided that there are a total of three types of ligands, chelators or bonding units. For example, a radiopharmaceutical comprised of one chelator or bonding unit from one reagent and two different ancillary or co-ligands is considered to be comprised of a ternary ligand system. Ancillary or co-ligands useful in the preparation of radiopharmaceuticals and in diagnostic kits useful for the preparation of said radiopharmaceuticals may be comprised of one or more oxygen, nitrogen, carbon, sulfur, phosphorus, arsenic, selenium, and tellurium donor atoms. A ligand can be a transfer ligand in the synthesis of a radiopharmaceutical and also serve as an ancillary or co-ligand in another radiopharmaceutical. Whether a ligand is termed a transfer or ancillary or co-ligand depends on whether the ligand remains in the radionuclide coordination sphere in the radiopharmaceutical, which is determined by the coordination chemistry of the radionuclide and the chelator or bonding unit of the reagent or reagents.

A “bacteriostat” is a component that inhibits the growth of bacteria in a formulation either during its storage before use of after a diagnostic kit is used to synthesize a radiopharmaceutical.

The term “bond”, as used herein, means either a single or double bond.

The term “bubbles” or “microbubbles,” as used herein, refers to vesicles which are generally characterized by the presence of one or more membranes or walls surrounding an internal void that is filled with a gas or precursor thereto. Exemplary bubbles or microbubbles include, for example, liposomes, micelles and the like.

A “carbohydrate” is a polyhydroxy aldehyde, ketone, alcohol or acid, or derivatives thereof, including polymers thereof having polymeric linkages of the acetal type.

A “chelator” or “bonding unit” is the moiety or group on a reagent that binds to a metal ion through the formation of chemical bonds with one or more donor atoms. Preferred chelators of the present invention are described in U.S. Pat. No. 6,511,648, the disclosure of which is hereby incorporated herein by reference in its entirety.

A “cyclodextrin” is a cyclic oligosaccharide. Examples of cyclodextrins include, but are not limited to, α-cyclodextrin, hydroxyethyl-α-cyclodextrin, hydroxypropyl-α-cyclodextrin, β-cyclodextrin, hydroxypropyl-β-cyclodextrin, carboxymethyl-β-cyclodextrin, dihydroxypropyl-β-cyclodextrin, hydroxyethyl-β-cyclodextrin, 2,6 di-O-methyl-β-cyclodextrin, sulfated-β-cyclodextrin, γ-cyclodextrin, hydroxypropyl-γ-cyclodextrin, dihydroxypropyl-γ-cyclodextrin, hydroxyethyl-γ-cyclodextrin, and sulfated γ-cyclodextrin.

A “diagnostic kit” or “kit” comprises a collection of components, termed the formulation, in one or more vials which are used by the practicing end user in a clinical or pharmacy setting to synthesize diagnostic radiopharmaceuticals. The kit preferably provides all the requisite components to synthesize and use the diagnostic pharmaceutical except those that are commonly available to the practicing end user, such as water or saline for injection, a solution of the radionuclide, equipment for heating the kit during the synthesis of the radiopharmaceutical, if required, equipment necessary for administering the radiopharmaceutical to the patient such as syringes, shielding, imaging equipment, and the like. Imaging agents are provided to the end user in their final form in a formulation contained typically in one vial, as either a lyophilized solid or an aqueous solution. The end user typically reconstitutes the lyophilized material with water or saline and withdraws the patient dose or just withdraws the dose from the aqueous solution formulation as provided.

The term “donor atom” refers to the atom directly attached to a metal by a chemical bond.

The suffix “ene” when used with the hydrocarbons defined above, indicates that the group has two points of attachment (i.e., is a diradical). For example, a saturated aliphatic hydrocarbon group disposed between two other moieties would be referred to herein as “alkylene.”

As used herein, the term “heterocycle” or “heterocyclic system” is intended to mean a stable 5- to 7-membered monocyclic or bicyclic or 7- to 10-membered bicyclic heterocyclic ring which is saturated, partially unsaturated, or unsaturated (aromatic), and which consists of carbon atoms and from 1 to 4 heteroatoms independently selected from the group consisting of N, O and S and including any bicyclic group in which any of the above-defined heterocyclic rings is fused to a benzene ring. The nitrogen and sulfur heteroatoms may optionally be oxidized. The heterocyclic ring may be attached to its pendant group at any heteroatom or carbon atom which results in a stable structure. The heterocyclic rings described herein may be substituted on carbon or on a nitrogen atom if the resulting compound is stable. If specifically noted, a nitrogen in the heterocycle may optionally be quaternized. It is preferred that when the total number of S and O atoms in the heterocycle exceeds 1, then these heteroatoms are not adjacent to one another. It is preferred that the total number of S and O atoms in the heterocycle is not more than 1. As used herein, the term “aromatic heterocyclic system” or “heteroaryl” is intended to mean a stable 5- to 7-membered monocyclic or bicyclic or 7- to 10-membered bicyclic heterocyclic aromatic ring which consists of carbon atoms and from 1 to 4 heteroatoms independently selected from the group consisting of N, O and S. It is preferred that the total number of S and O atoms in the aromatic heterocycle is not more than 1. Examples of heterocycles include, but are not limited to, 1H-indazole, 2-pyrrolidonyl, 2H,6H-1,5,2-dithiazinyl, 2H-pyrrolyl, 3H-indolyl, 4-piperidonyl, 4aH-carbazole, 4H-quinolizinyl, 6H-1,2,5-thiadiazinyl, acridinyl, azocinyl, benzimidazolyl, benzofuranyl, benzothiofuranyl, benzothiophenyl, benzoxazolyl, benzthiazolyl, benztriazolyl, benztetrazolyl, benzisoxazolyl, benzisothiazolyl, benzimidazalonyl, carbazolyl, 4aH-carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, isobenzofuranyl, isochromanyl, isoindazolyl, isoindolinyl, isoindolyl, isoquinolinyl, isothiazolyl, isoxazolyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl., oxazolyl, oxazolidinylperimidinyl, phenanthridinyl, phenanthrolinyl, phenarsazinyl, phenazinyl, phenothiazinyl, phenoxathiinyl, phenoxazinyl, phthalazinyl, piperazinyl, piperidinyl, pteridinyl, piperidonyl, 4-piperidonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, pyrrolyl, quinazolinyl, quinolinyl, 4H-quinolizinyl, quinoxalinyl, quinuclidinyl, carbolinyl, tetrahydrofuranyl, tetrahydroisoquinolinyl, tetrahydroquinolinyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, xanthenyl. Preferred heterocycles include, but are not limited to, pyridinyl, furanyl, thienyl, pyrrolyl, pyrazolyl, imidazolyl, indolyl, benzimidazolyl, 1H-indazolyl, oxazolidinyl, benzotriazolyl, benzisoxazolyl, oxindolyl, benzoxazolinyl, or isatinoyl. Also included are fused ring and spiro compounds containing, for example, the above heterocycles.

As used herein, the term “lipid” refers to a synthetic or naturally-occurring amphipathic compound which comprises a hydrophilic component and a hydrophobic component. Lipids include, for example, fatty acids, neutral fats, phosphatides, glycolipids, aliphatic alcohols and waxes, terpenes and steroids. Exemplary compositions which comprise a lipid compound include suspensions, emulsions and vesicular compositions.

“Liposome” refers to a generally spherical cluster or aggregate of amphipathic compounds, including lipid compounds, typically in the form of one or more concentric layers, for example, bilayers. They may also be referred to herein as lipid vesicles.

A “lyophilization aid” is a component that has favorable physical properties for lyophilization, such as the glass transition temperature, and is generally added to the formulation to improve the physical properties of the combination of all the components of the formulation for lyophilization.

“Metallopharmaceutical” means a pharmaceutical comprising a metal. The metal is the cause of the imageable signal in diagnostic applications. Radiopharmaceuticals are metallopharmaceuticals in which the metal is a radioisotope.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, “pharmaceutically acceptable salts” refer to derivatives of the disclosed compounds modified by making acid or base salts. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like. The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, p. 1418, the disclosure of which is hereby incorporated by reference.

A “polyalkylene glycol” is a polyethylene glycol, polypropylene glycol, polybutylene glycol, or similar glycol having a molecular weight of less than about 5000, terminating in either a hydroxy or alkyl ether moiety.

As used herein, the term “polycarboxyalkyl” means an alkyl group having from about two and about 100 carbon atoms and a plurality of carboxyl substituents; and the term “polyazaalkyl” means a linear or branched alkyl group having from about two and about 100 carbon atoms, interrupted by or substituted with a plurality of amine groups.

By “reagent” is meant a compound of this invention capable of direct transformation into a metallopharmaceutical of this invention. Reagents may be utilized directly for the preparation of the metallopharmaceuticals of this invention or may be a component in a kit of this invention.

A “reducing agent” is a compound that reacts with a radionuclide, which is typically obtained as a relatively unreactive, high oxidation state compound, to lower its oxidation state by transferring electron(s) to the radionuclide, thereby making it more reactive. Reducing agents useful in the preparation of radiopharmaceuticals and in diagnostic kits useful for the preparation of said radiopharmaceuticals include, for example, stannous chloride, stannous fluoride, formamidine sulfamic acid, ascorbic acid, cysteine, phosphines, and cuprous or ferrous salts. Other reducing agents are described, for example, in Brodack et. al., PCT Application 94/22496, the disclosure of which is incorporated herein by reference in its entirety.

The term “salt”, as used herein, is used as defined in the CRC Handbook of Chemistry and Physics, 65th Edition, CRC Press, Boca Raton, Fla., 1984, as any substance which yields ions, other than hydrogen or hydroxyl ions.

A “stabilization aid” is a component that is typically added to the metallopharmaceutical or to the diagnostic kit either to stabilize the metallopharmaceutical or to prolong the shelf-life of the kit before it must be used. Stabilization aids can be antioxidants, reducing agents or radical scavengers and can provide improved stability by reacting preferentially with species that degrade other components or the metallopharmaceutical.

By “stable compound” or “stable structure” is meant herein a compound that is sufficiently robust to survive isolation to a useful degree of purity from a reaction mixture, and formulation into an efficacious pharmaceutical agent.

A “solubilization aid” is a component that improves the solubility of one or more other components in the medium required for the formulation.

The term “substituted”, as used herein, means that one or more hydrogens on the designated atom or group is replaced with a selection from the indicated group, provided that the designated atom's or group's normal valency is not exceeded, and that the substitution results in a stable compound. When a substituent is keto (i.e., ═O), then 2 hydrogens on the atom are replaced.

A “transfer ligand” is a ligand that forms an intermediate complex with a metal ion that is stable enough to prevent unwanted side-reactions but labile enough to be converted to a metallopharmaceutical. The formation of the intermediate complex is kinetically favored while the formation of the metallopharmaceutical is thermodynamically favored. Transfer ligands useful in the preparation of metallopharmaceuticals and in diagnostic kits useful for the preparation of diagnostic radiopharmaceuticals include, for example, gluconate, glucoheptonate, mannitol, glucarate, N,N,N′,N′-ethylenediaminetetraacetic acid, pyrophosphate and methylenediphosphonate. In general, transfer ligands are comprised of oxygen or nitrogen donor atoms.

As used herein, the term “vesicle” refers to a spherical entity which is characterized by the presence of an internal void. Preferred vesicles are formulated from lipids, including the various lipids described herein. In any given vesicle, the lipids may be in the form of a monolayer or bilayer, and the mono- or bilayer lipids may be used to form one of more mono- or bilayers. In the case of more than one mono- or bilayer, the mono- or bilayers are generally concentric. The lipid vesicles described herein include such entities commonly referred to as liposomes, micelles, bubbles, microbubbles, microspheres and the like. Thus, the lipids may be used to form a unilamellar vesicle (comprised of one monolayer or bilayer), an oligolamellar vesicle (comprised of about two or about three monolayers or bilayers) or a multilamellar vesicle (comprised of more than about three monolayers or bilayers). The internal void of the vesicles may be filled with a liquid, including, for example, an aqueous liquid, a gas, a gaseous precursor, and/or a solid or solute material, including, for example, a bioactive agent, as desired.

As used herein, the term “vesicular composition” refers to a composition which is formulate from lipids and which comprises vesicles.

As used herein, the term “vesicle formulation” refers to a composition which comprises vesicles and a bioactive agent.

The following abbreviations are used herein:

| | |
|---|---|
| Acm | acetamidomethyl |
| b-Ala, beta-Ala or | 3-aminopropionic acid |
| bAla | |
| ATA | 2-aminothiazole-5-acetic acid or 2-aminothiazole-5- |
| | acetyl group |
| Boc | t-butyloxycarbonyl |
| CBZ, Cbz or Z | Carbobenzyloxy |
| Cit | citrulline |
| Dap | 2,3-diaminopropionic acid |
| DCC | dicyclohexylcarbodiimide |
| DIEA | diisopropylethylamine |
| DMAP | 4-dimethylaminopyridine |
| DCM | dichloromethane |
| DMSO | dimethyl sulfoxide |
| DMF | N,N-dimethylformamide |
| EtOAc | Ethyl Acetate |
| EOE | ethoxyethyl |
| Fmoc | 9-fluorenylmethoxycarbonyl |
| HBTU | 2-(1H-Benzotriazol-1-yl)-1,1,3,3-tetramethyluronium |
| | Hexafluorophosphate |
| hynic | boc-hydrazinonicotinyl group or 2-[[[5-[carbonyl]-2- |
| | pyridinyl]hydrazono]methyl]-benzenesulfonic acid, |
| NMeArg or | a-N-methyl arginine |
| MeArg | |
| NMeAsp | a-N-methyl aspartic acid |
| NMM | N-methylmorpholine |
| NMP | N-Methyl-2-pyrrolidone |
| OcHex | O-cyclohexyl |
| OBzl | O-benzyl |
| oSu | O-succinimidyl |
| TBAF | tetrabutylammonium fluoride |
| TBTU | 2-(1H-Benzotriazol-1-yl)-1,1,3,3-tetramethyluronium |
| | tetrafluoroborate |
| THF | tetrahydrofuranyl |
| THP | tetrahydropyranyl |
| Tos | tosyl |
| Tr or Trt | trityl |

The present invention is further described in the following non-limiting examples.

### EXAMPLES

### Example 1

### [¹⁸F]-5′Fluoroadenosine

### 2′,3′ isoproylidene adenosine

A 250 ml round bottom flask was charged with 10 gm adenosine and to it was added 100 ml acetone. Methanesulfonyl chloride (10 ml) was then added drop-wise to the above solution. The solution was stirred for 30 minutes, after which time 100 ml of 1M Na₂CO₃ was carefully added. This sample was stirred for another 30 minutes and the solution filtered. The filtrate was taken up in dichloromethane, washed with water, and dried. The organic solvent was removed to afford the 4.45 grams pure 2′,3′ isopropylidene adenosine as the product.

### 5′-Tosyloxy 2′,3′-isopropylidene adenosine

To a 100 ml flask was added 250 mg of the isopropylidene adenosine. To this sample was added THF (3 ml) followed by NaH (29.2 mg). This was stirred for 20 minutes at room temperature after which p-toluenesulfonyl chloride was added in one lot (232 mg). The mixture was stirred for 20 minutes after which it was deemed complete by LC-MS (Liquid Chromatography-Mass Spectrometry). The organic solvent was removed in vacuo and the crude mixture subjected to purification by silica gel chromatography (ethyl acetate:dichloromethane) to afford 215 mg of the pure product.

### 5′-Fluoro 2′,3′-isopropylidene adenosine

A 50 ml round bottom flask was charged with 150 mg of 5′-Tosyloxy 2′,3′-isopropylidine adenosine. To this was added 3.25 ml of tetrabutylammonium fluoride (TBAF) solution (1M in THF) and the reaction mixture was stirred at room temperature for 15 minutes followed by refluxing at 120° C. for 15 minutes. All organic solvent was removed and the crude product was purified by flash chromatography (ethyl acetate: dichloromethane) to obtain 60 mg of pure product.

### 5′-Fluoroadenosine

To 42 mg of 5′-Fluoro 2′,3′-isopropylidene adenosine was added 1.5 ml of 90% formic acid. The reaction mixture was stirred for 2 hours after which it is complete. This sample was then purified by HPLC (Luna C18 column, Flow rate=20 ml/min; Gradient: 2-100% mobile phase B in 12 minutes; A=0.1% trifluoroacetic acid in water and B=0.1% trifluoroacetic acid in 90% acetinitrile) to obtain 32 mg of pure product.

### [¹⁸F]-5′Fluoro 5′-deoxyadenosine

10 mg of 5′-tosyloxy 2′,3′-isopropylidene adenosine in 0.5 ml of tetrahydrofuran was added to [¹⁸F]-tetrabutylammonium fluoride in tetrahydrofuran (0.42-5.41 GBq, 11.3-146.2 mCi). The reaction mixture was incubated at 100° C. for 20 minutes and then evaporated to dryness. To the above mixture was then added 0.5 ml of 90% formic acid and the reaction mixture incubated for 15 minutes at 50° C. The residue was then evaporated to dryness under a stream of nitrogen and redissolved in 1.0 ml of water. The mixture was filtered by a syringe filter and the filtrate was purified by preparative reverse phase column using method described below.

Method A: 2-100% mobile phase B over 12 minutes . Mobile phase A: 0.1% TFA in water; Mobile phase B: 0.1% TFA in 90% acetonitrile

### Example 2

### 2-Aminoadensosine

### 2-aminoadenosine

To 1.0 gm of dry guanosine (7.06 mmol) was added dry pyridine and this was cooled to 0° C. in a ice bath. This was followed by drop-wise addition of trifluoroacetic anhydride (4.9 ml, 35.3 mmol). The above solution was stirred for 30 minutes after which an additional 2.5 ml trifluoroacetic anhydride was added and the solution was stirred for an additional 30 minutes. 20 ml of cold, concentrated aqueous ammonia was then added to the above solution and stirring was continued for an additional 1.5 hrs, after which the mixture was evaporated to dryness and the residue dissolved in water and purified using reversed phase HPLC (Luna C18;; Flow rate=80 ml/min; Gradient: 0-15% mobile phase B over 40 minutes; Mobile phase A=0.1% trifluoroacetic acid in water and Mobile phase B=0.1% trifluoroacetic acid in 90% acetonitrile). This yielded 510 mg of 2-amino adenosine as the pure product.

### 2-Fluoroadenosine

To a stirred solution of 0.15 g (0.53 mmol) 2-aminoadenosine in 0.5 ml 56% HF/pyridine cooled to −10° C. was added a concentrated solution of 0.05 g of KNO₂ (0.57 mmol) in water drop-wise. Stirring was continued for 2 hours after which the mixture was poured on a stirred ice cold slurry of 1 g of powdered CaCO₃ in water. After letting stand overnight the solution was filtered and the filtrate evaporated to afford 2-Fluoroadenosine. This was washed with water, ethanol and methyl-tert-butyl ether and recrystallized from ethanol to afford 120 mg of pure 2-Fluoroadenosine.

### Example 3

### [¹⁸F]-2-Fluoroadenosine

A solution of [¹⁸F]-tetrabutylammonium fluoride in tetrahydrofuran (1-100 mCi) is added to 20 mg of 2-N,N,N-(trimethylammonium)adenosine triflate. This sample is incubated at 50° C. for 20 minutes after which the solvent evaporates. To obtain the title compound, the residue is purified by preparative reverse phase chromatography using the following method: 0-15% Mobile phase B over 40 minutes. Mobile phase A: 0.1% TFA in water; Mobile phase B: 0.1% TFA in 90% acetonitrile

The disclosures of each patent, patent application, and publication cited or described in this document are hereby incorporated herein by reference, in their entireties.

Various modifications of the invention, in addition to those described herein, will be apparent to those skilled in the art from the foregoing description. Such modifications are also intended to fall within the scope of the appended claims.

1. An agent comprising
(a) an adenosine analog moiety or an adenosine moiety, and
(b) an imaging moiety.

2. The agent of claim 1 wherein the adenosine analog moiety or the adenosine moiety is covalently linked to the imaging moiety.

3. The agent of claim 1 wherein the adenosine analog moiety or the adenosine moiety is linked to the imaging moiety via a non-covalent interaction.

4. The agent of claim 2 having the formula (I) 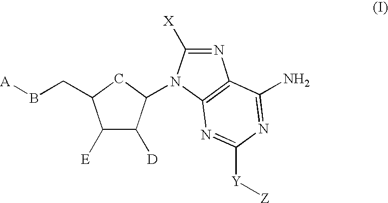
wherein
A is an imaging moiety or H;
B is a direct bond, O, alkylene, arylene, or alkylene ether;
C is O, N(R₁), or CH(R₁), wherein R₁ is H or C₁-C₆ alkyl;
D is an imaging moiety, F, OH, or H;
E is an imaging moiety, F, OH, or H;
X is an imaging moiety or H;
Y is a direct bond, alkylene, arylene, or alkylene ether; and
Z is an imaging moiety or H;
with the proviso that at least one of A, D, E, X, and Z is an imaging moiety and the agent is not [¹⁸F]fluorodeoxyadenosine.

5. The agent of claim 4 wherein X is H.

6. The agent of claim 4 wherein Y is a direct bond.

7. The agent of claim 4 wherein B is a direct bond and A is an imaging moiety.

8. The agent of claim 4 wherein B is O and A is H.

9. The agent of claim 4 wherein B is an alkylene ether.

10. The agent of claim 9 wherein B is a C₁-C₆ alkylene ether.

11. The agent of claim 9 wherein B is ethyl ether.

12. The agent of claim 2 having a formula (II): 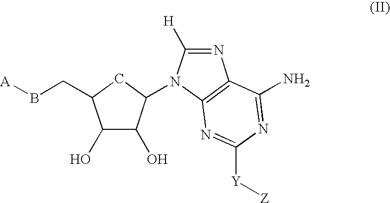
wherein
A is an imaging moiety or H;
B is a direct bond or O;
Y is a direct bond, alkylene, arylene, or alkylene ether; and
Z is an imaging moiety or H;
with the proviso that at least one of A and Z is an imaging moiety.

13. The agent of claim 12 wherein Y is a direct bond.

14. The agent of claim 2 having a formula (III): 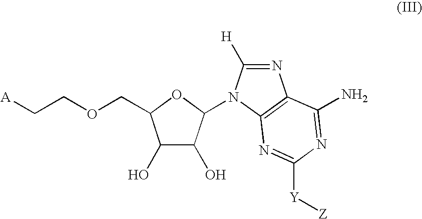
wherein
A is an imaging moiety or H;
Y is a direct bond, alkylene, arylene, or alkylene ether; and
Z is an imaging moiety or H;
with the proviso that at least one of A and Z is an imaging moiety.

15. The agent of claim 14 wherein Y is a direct bond.

16. The agent of claim 1 wherein the imaging moiety is a radioisotope for nuclear medicine imaging, a paramagnetic species for use in MRI imaging, an echogenic entity for use in ultrasound imaging, a fluorescent entity for use in fluorescence imaging, or a light-active entity for use in optical imaging.

17. The agent of claim 16, wherein the radioisotope for nuclear medicine imaging is selected from the group consisting of ¹¹C, ¹³N, ¹⁸F, ¹²³I, ¹²⁵I, ^{99m}Tc, 95Tc, ¹¹¹In, ⁶²Cu, ⁶⁴Cu, ⁶⁷Ga, and ⁶⁸Ga.

18. The agent of claim 17, wherein the imaging moiety is ¹⁸F.

19. The agent of claim 16, wherein the paramagnetic species for use in MRI imaging is a radioisotope selected from the group consisting of Gd³⁺, Fe³⁺, In³⁺, and Mn²⁺.

20. The agent of claim 16, wherein the echogenic entity for use in ultrasound imaging is a fluorocarbon encapsulated surfactant microsphere.

21. The agent of claim 4, wherein:
A is ¹⁸F or OH;
B is ethyl ether;
C is O;
D and E are each OH;
X is H;
Y is a direct bond; and
Z is H or ¹⁸F,
with the proviso that at least one of A and Z is ¹⁸F.

22. The agent of claim 2, wherein the agent is:

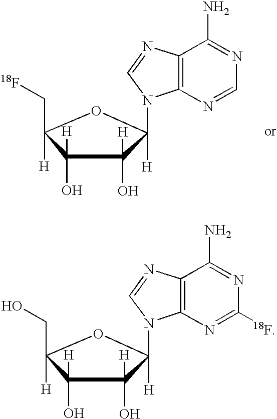

23. A method of imaging myocardial perfusion, comprising:
administering to a subject an agent according to claim 1; and
scanning the subject using diagnostic imaging to detect areas of greater agent concentration.

24. The method of claim 23 wherein the agent is described by claim 2.

25. The method of claim 23 wherein the agent is described by claim 3.

26. The method of claim 23 wherein the agent is described by formula (I) 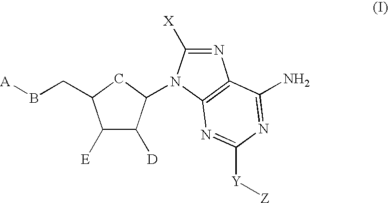
wherein
A is an imaging moiety or H;
B is a direct bond, O, alkylene, arylene, or alkylene ether;
C is O, N(R₁), or CH(R₁), wherein R₁ is H or C₁-C₆ alkyl;
D is an imaging moiety, F, OH, or H;
E is an imaging moiety, F, OH, or H;
X is an imaging moiety or H;
Y is a direct bond, alkylene, arylene, or alkylene ether; and
Z is an imaging moiety or H;
with the proviso that at least one of A, D, E, X, and Z is an imaging moiety.

27. The method of claim 23 wherein the agent is described by formula (II): 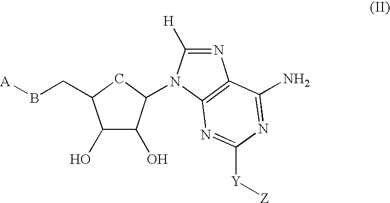
wherein
A is an imaging moiety or H;
B is a direct bond or O;
Y is a direct bond, alkylene, arylene, or alkylene ether; and
Z is an imaging moiety or H;
with the proviso that at least one of A and Z is an imaging moiety.

28. The method of claim 23 wherein the agent is described by formula (III): 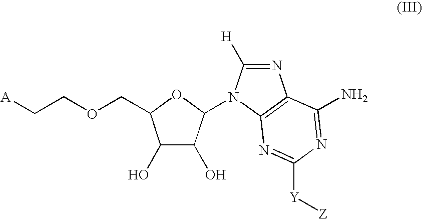
wherein
A is an imaging moiety or H;
Y is a direct bond, alkylene, arylene, or alkylene ether; and
Z is an imaging moiety or H;
with the proviso that at least one of A and Z is an imaging moiety.

29. The method of claim 23 wherein the agent is:

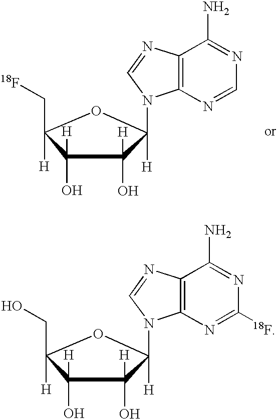

30. A kit for myocardial perfusion imaging comprising
(a) a compound A which comprises an adenosine moiety or an adenosine analog moiety and
(b) a compound B which comprises an imaging moiety;
wherein Compounds A and B can be reacted to each other and form the agent of claim 1.
